(19) United States
(12) Patent Application Publication
(10) Pub. No.: US 2012/0184030 A1
(43) Pub. Date: Jul. 19, 2012

(54) METHODS FOR ESTABLISHING SYMBIOSES

(76) Inventors: Zoran Dermanovic, Zagreb (HR); Miroslav Radman, Gentilly (FR)

(21) Appl. No.: 13/384,415

(22) PCT Filed: Jul. 19, 2010
(86) PCT No.: PCT/FR2010/051518

§ 371 Date: Apr. 6, 2012

(30) Foreign Application Priority Data
Jul. 17, 2009 (FR) 0954994

(51) Int. Cl. C12N 5/0735; C12N 1/16; C12N 1/18; C12N 5/071

(52) U.S. Cl. 435/354; 435/325; 435/255.2

(57) ABSTRACT

The present invention relates to methods for establishing a symbiosis, including the following steps: selecting an organism or an organelle to constitute the symbiont and an organism to constitute the host, the latter not existing naturally in a symbiotic relationship; contacting the symbiont and the host; and maintaining the combination of the symbiont and the host.

### TECHNICAL FIELD

This invention concerns establishing new symbiotic relationships between one organism and another or between an organelle and an organism.

The new symbiotic relationships thus established may have many applications, particularly from the bioenergetic, therapeutic or environmental points of view.

### PRIOR ART

After life emerged on Earth about 4 billion years ago, the most important event which allowed the evolution of complex life forms was the formation of stable hereditary endosymbioses. The first eukaryote cells thus established a symbiotic relationship with the proteobacteria, which resulted in the development of mitochondria. A second symbiotic event created photosynthesis, when the cyanobacteria produced to endosymbiotic photosynthetic organelles (cyanelles and plasts) about 1.5 billion years ago.

It seems improbable that such an event only occurred once. Indeed, recent studies have shown that the formation of endosymbioses, as well as the resulting horizontal transfer of genes to the cell nucleus, could well be one of the main motors of continuing evolution.

Such examples can be found in bacterial biotopes present in geothermal springs (an austere, competitive environment, poor in nutrients), as well as in the development of new non-hereditary ‘klepto-endosymbiotic’ relationships, such as in the sea slug Elysia chlorotica.

The parallel evolution of species has also created complex networks of exosymbiotic, commensal and parasitic relationships in multicellular or monocellular organisms, in any combination of these, with a neutral, beneficial or adverse effect on the host. On this basis and according to R. Dawkins' ‘extended phenotype’ proposal, the genes of an organism (the parasite/symbiont/commensal organism) have phenotypic effects on another organism (the host organism).

Given the evolutionary success of ancient endosymbiotic events, namely the evolution of plants, fungi and animals, it can be said that the positive selection pressure for the results of recent endosymbiotic events is relatively low. In addition, given the necessity of capturing the potential symbiont or being infected by it, not digesting it, not dying because of it and of preserving it in a functional form in the cytosol, the probability of establishing new endosymbiotic relationships in natural populations is also very low.

What is more, some metabolic pathways, or certain metabolites resulting from them, are of great interest but remain unusable due to characteristics of the host organism, for example the said organism's growing conditions.

The question of energy is one of humanity's current problems. It is a strategic priority for countries with rapid economic development, those with a high population and industrialised countries with few natural resources to ensure that they have abundant, ecological and viable energy resources.

Apart from the harmful emission of CO₂ (with all its consequences for atmospheric pollution and global warming), oil and to some extent coal, which are currently the main sources of energy, are subject to unpredictable fluctuations, sometimes politically motivated, which affect their price and market availability. All these factors make them items of strategic importance for any country, particularly any industrialised country, and particularly the major industrialised countries.

Hydro-electric energy, which requires major investment for construction and maintenance, is not cheap enough and often has a negative impact on the biotopes of rivers.

The renewable ecological sources of energy (wind, photovoltaic cells, tides, etc.) are still only a minor part of energy supply and in many cases, latest developments are seriously limiting their optimal economic use.

Consequently, one of the most promising, ecological and viable prospects in the field of energy is the biotechnological creation of microorganisms capable either of producing oil (ethanol or butanol) by photosynthesis or, in the case of economically important animals, of creating those animals that can directly use the sun's energy.

The production of ethanol which can be used as a fuel is a promising pathway which, despite various technical solutions proposed in documents U.S. Pat. No. 4,242,455, U.S. Pat. No. 4,350,765, U.S. Pat. No. 4,413,058, WO 88/09379, U.S. Pat. No. 6,699,696, or U.S. Pat. No. 5,550,050, cannot be exploited on a large scale.

In another technical field, i.e. medicine, the treatment of certain diseases requires regularly and repeatedly taking molecules which are difficult and/or costly to synthesise. This is the case for example for metabolic or endocrinological diseases in which one or more vital enzyme or other bioactive substance is missing through lack of intrinsic production.

There therefore seems to be a continuous need to develop new technical solutions which will allow natural metabolites present in certain organisms to be exploited and thus produce useful metabolites.

### DESCRIPTION OF THE INVENTION

The present invention is based on the demonstration by the Applicant of a method establishing new, ‘forced’, and controlled symbiosis.

‘Symbiosis’ is taken to mean a close, temporary (non-hereditary) or lasting (hereditary) association between two organisms of different species, one being considered as the symbiont and the other, in general the larger, as the host. The host provides the nutritional environment to the smaller endosymbiont. Without being limiting, this also includes cell organelles resulting from previous endosymbiotic events, as well as organelles created artificially, and modified prokaryotes or eukaryotes designed to function inside living cells as ‘artificial organelles’.

The issue consists therefore of associating two organisms or an organism and an organelle, which do not pre-exist in this form or in this type of relationship in nature.

The sharing by the organisms involved in the symbiosis of capacities, in particular metabolic capacities, may have many advantages, particularly for the production of intermediate and final metabolites. These new symbiotic relationships may also give rise to therapeutic, or more generally beneficial effects.

Hosts will be obtained which are ‘boosted’ by the symbiosis, i.e. an organism with an improved state of health, or more generally one whose phenotype has been changed by the introduction and possibly by the modification (genetic or in another way) of its symbiont, commensal or parasite.

‘Symbiont’ is taken to mean an organism or organelle which lives in symbiosis with another organism, for their mutual benefit.

‘Commensal’ is taken to mean an organism which lives on or with another organism without providing it with any benefits but without harming it.

‘Parasite’ is taken to mean an organism which lives and develops at the expense of another organism, without taking into account its pathogenicity.

In the rest of the description and within the framework of this invention, the terms ‘symbiont’ and ‘symbiotic relationship’ are used as generic terms to cover these different situations.

The ultimate objective consists in forming improved, altered or entirely new relationships between two organisms or an organism and an organelle, constituting respectively the host and the symbiont. The present invention therefore offers the possibility of manipulating (reducing, increasing or modifying) symbiotic relationships, as regards their pathogenicity and/or their physiological and/or behavioural effect, in order to serve a specific therapeutic and/or diagnostic and/or biotechnological and/or bioactive purpose. The present invention thus offers an alternative to the chemical compounds classically used in these different areas.

In practice, a method according to the invention comprises at least the following steps:
Selection of an organism or organelle intended to act as the symbiont and of an organism intended to be the host, these two entities not naturally existing in a symbiotic relationship;
Possible genetic modification to an organism or an organelle acting as the symbiont and/or of the organism forming the host;
Bringing the symbiont and the host together;
Maintaining the association between the symbiont and the host.

The symbiont consists of a whole organism or organelle, i.e. a differentiated intracellular unit.

‘Organism’ is taken to mean viruses and/or bacteria and/or protozoa and/or prokaryotes and/or mono or multicellular eukaryotes. To advantage, the organism is not selected from within the following group: a human, an animal or a human embryonic stem cell.

As already stated, this method brings together two entities, the symbiont and the host. These pairs are selected case by case, depending particularly on their compatibility and the interest of their respective phenotypes.

Moreover, according to a characteristic of the invention, the selected pair does not form a symbiosis in nature following a spontaneous natural event. Natural symbioses are widely documented.

To advantage according to the invention, the symbiont is not a nitrogen-fixing bacterium or an endophytic fungus, since this type of symbiosis has already been reported in the prior art.

As concerns organelles, mitochondria originating from the same species as the host are also to advantage excluded. Similarly, mitochondria from the same species as the host which have been modified by up to 10% of their sequence and thus have sequences which are more than 90% identical are to advantage excluded.

On the other hand, the organelles could be cyanelles, artificial mitochondria, chloroplasts, hydrogenosomes, or mitochondria xenotransplanted from a species different to that of the host.

‘Genetic modification’ is taken to mean the fact that the genetic identity of the symbiont and/or the host is modified, particularly to confer on it a phenotype of particular interest or to encourage the establishment of symbiosis. In the context of the invention, genetic modification of the host and/or the symbiont is to advantage intended to contribute to the establishment and/or maintenance of the symbiosis.

More precisely for this invention, the term ‘genetic modification’ is defined as a qualitative and/or quantitative change in the total DNA content of the symbiont and/or of the host: nucleus+organelles+free cytoplasmic nucleotides and plasmids.

Such genetic modification can result particularly from mutagenesis, for example by exposure to UV radiation or by being put into the presence of a mutagenic agent, or by transfer (insertion) of genetic material. All these techniques are well known to those working in the field.

Following this genetic modification, to advantage made before the symbiont and the host come into contact, it is generally necessary to screen in order to select the organisms or organelles which have actually undergone this change. The screening may particularly depend on the desired phenotype or on a marker introduced for this purpose.

As already stated, the host, as well as the symbiont, may be subjected to such genetic modifications, using similar techniques. Any host may be subjected to this procedure.

In a particular embodiment, the two partners—the symbiont and the host—are subjected to this genetic modification.

The second crucial step is to put the symbiont and the host into contact with the aim of establishing symbiosis, which may be either endosymbiosis or exosymbiosis.

‘Endosymbiosis’ is taken to mean symbiosis in which one of the two organisms lives inside the cell or cells of the other.

‘Exosymbiosis’ is taken to mean symbiosis in which the two organisms live in close proximity to each other. The symbiont is found inside or outside the cells of one or of all of the organs or systems of organs of the host.

The purpose of this step is to create experimental conditions promoting the initial ‘capture’ of the potential symbionts.

There are various techniques of establishing contact which are well known to those working in the field. The technique can be chosen from among the following: insertion, fusion, in particular cell fusion, implantation, micro-injection, electroporation, stimulated or natural phagocytosis, cross-species fertilisation, infection, attenuation.

The symbiont/host pair is to advantage subjected simultaneously or successively to selection and/or maintenance pressure. Experimental conditions are created exerting strong positive selection pressure to establish and/or maintain the new symbiotic relationships. Such conditions are chosen for example from the group including:
the presence of toxic substances and/or antibiotics (e.g. for metabolic efficacy and/or recycling waste material—bioremediation, and/or the selection of resistant mutants);
light dependent growth (e.g. for photosynthesis);
irradiation/desiccation (e.g. for robustness);
nutritional deficit/reduction and/or dietary modification (e.g. for metabolic efficacy and/or dietary adaptation);
selection of the host for resistance present in or carried by the symbiont (e.g. resistance to an antibiotic or a toxin);
selection from a large population (to increase the chance of selecting a rare event).

This method thus allows totally new organisms to be created, which can equally well be microorganisms, fungi or multicellular animal organisms.

There are many applications, particularly related to the introduction of photosynthetic activity, i.e. the possibility for organisms other than plants to create energy-rich organic carbohydrates, solely from sun, water and air.

For this application, partners preferred are:
For the symbiont: Chlorobium tepidum, a large, anaerobic, obligate autotroph; cyanelles from Cyanophora paradoxa; Ostreococcus tauri, a smaller planktonic eukaryote with good photosynthetic activity; or Rhodobacter sphaeroides, an autotrophic bacterium;
For the host: frog zygotes (Xenopus tropicalis albinos); albino nude mice embryonic stem cells; Saccharomyces cerevisiae (brewer's yeast) or Saccharomyces uvarum (large yeast cells).

### Examples of Embodiments

The way in which the invention can be carried out and the advantages which follow from it are well illustrated by the examples of embodiments below, which are in no way limiting.

### I/ Protocols For Establishing Symbiosis

### 1/ Experimental Conditions

Conditions Favouring the Initial Introduction of the Endosymbiont into the Host:
Microinjection
Cell fusion
Electroporation
Stimulated or natural phagocytosis
Cross-species fertilisation
Infection
Mutagenesis of the potential endosymbiont
Attenuation

Conditions Creating Positive Selection Pressure to Establish and Maintain New Symbiotic Relationships:
Deficiency/presence of toxic substances (e.g. for metabolic efficacy and/or recycling waste materials—bioremediation)
Light dependent growth (e.g. for photosynthesis)
Irradiation/desiccation (e.g. for robustness)
Selection of the host for resistances carried by the endosymbiont (e.g. resistance to antibiotics, resistance to a toxin, etc.)
Selection from within large populations (to increase the chance of selecting a rare event)

Endosymbionts:
1) Chlorobium tepidum (large, anaerobic, obligate autotroph)
2) Cyanelles of Cyanophora paradoxa (5 to 10% of the genome of the cyanobacteria)
3) Ostreococcus tauri (smaller eukaryote, a good photosynthesiser)

Hosts:
a) Frog zygotes (Xenopus tropicalis albinos)
b) Albino nude mice embryonic stem cells
c) Saccharomyces cerevisiae (brewer's yeast)
d) Saccharomyces uvarum (the largest yeast)

### 2/ Preliminary Tests

### 2-1/ Sonication of Ostreococcus tauri; Examination of the Degree of Membrane Destruction

The cell suspension was put on ice and subjected to short, fixed bursts of sonication.
After each burst, the main test tube was cooled again.
After each burst, a sample was taken with a micropipette and tested under the microscope to determine the optimal ratio between the cell membranes subjected to the bursts and the intact plastids.
The optimal protocol was pre-treatment of Ostreococcus before microinjection.

### 2-2/ Lysozyme Pre-Treatment of C. paradoxa

Preparation of lysozyme solutions at concentration of 5, 20, 100, 200 and 300 μg/ml;
Determination of the standard concentration of donor cells in the suspension;
Standard temperatures=20, 25, 30, 37° C.;
Standard time=20 minutes incubation, then washing by centrifugation and resuspension;
Test under the microscope for the collapse of the cell membranes and the absence of flagella;
Minimum of 20 tests=5 concentrations at 4 temperatures;
The optimal protocol was pre-treatment of C. paradoxa before microinjection.

### 2-3/ Lysozyme Pre-Treatment of O. tauri

As in 2-2

### 2-4/ Pre-treatment of Yeast Cultures with Hydroxyurea (for Large Yeasts Cells)

Media:

YEP-galactose-PVP medium composed of 15 g of yeast extract, 30 g of bacteriological peptone, 30 g of galactose, 280 g of polyvinylpyrrolidone (PVP-40, Sigma) and 50 μg of adenine in 1 l of distilled water. The medium was sterilised by autoclaving. The hydroxyurea (HU) was provided by Sigma. The synthetic factor α was obtained from the Peptide Institute, Osaka, Japan.
Concentration=1.5 mg/ml HU;
Exposure time=112 minutes;
Standardised number of cells in suspension;
After exposure, washing by centrifugation and resuspension in a medium not containing HU.

The largest yeast cells were candidates for microinjection.

### 2-5/ Suppression of Genes Encoding Two Essential Amino Acids (Limitation of the Biological Danger)

Deletion of the HIS-3 and LEU-2 genes;
(for Saccharomyces cerevisiae, performed at MedILS).
2-6/ Pre-treatment by UV Mutagenesis for all the Donors (Possibly with Ethyl-methyl-sulphonate or EMS):
UV lamp+UV dosimeter (objective: 10% of survivors; after the latent period, the surviving cells were the donors which, after fragilisation pre-treatment, received the microinjection);
Standardised cell concentration;
Mobility test under the microscope for viability (Cyanophora paradoxa) in a standardised grid;
Dihydrorhodamine staining for viability (Ostreococcus tauri, Chlorobium tepidum);
Latent period=1-2 generations (doubling, number of survivors quadrupled).

### 2-7/ Test of the Toxicity of the Donor's Nutrient Medium for the Host

By sham injection of the medium only; if it was toxic, centrifugation of the donor cells and resuspension in a 0.9% NaCl solution.

### 3/ Experiments

### A/ Donor 1—Host a

Standardised viable anaerobic culture of Chlorobium tepidum;
Irradiation of the donor (see preliminary tests above);
Following a latent period of 4 hours (time for the number of survivors to quadruple, 2 generations), microinjection into a frog 1-cell zygote;
2 doses: a) 10-15 donor cells; b) 5-10% of the volume of the host cells: (culture of surviving donors after a latent period).

Observations:
1^{st} observation—32 cell stage
2^{nd} observation—tadpole (gills)
3^{rd} observation—tadpole (lungs)
4^{th} observation—adult frog (in vivo, histological analysis)

### B/ Donor 1—Host b

Standardised viable anaerobic culture of Chlorobium tepidum;
Standardised viable culture of albino nude mice embryonic stem cells
Irradiation of the donor (see preliminary tests above);
After a latent period of 4 hours (time for the number of survivors to quadruple, 2 generations), polyethylene glycol (PEG) induced fusion with mouse embryonic stem cells (donor: host ratio=5-10:1);
After fusion, electrical stimulation of membrane fusion (1 kV/cm of the depth of the culture, 2 pulses).

Observations:
After fusion, all the cultures were placed under lighting (sunlight spectrum) with a circadian rhythm (12 h of light: 12 h of darkness)
1^{st} observation—1^{st} day (microscope, presence of cyanobacteria in the cytosol, presence of chlorophyll)
2^{nd} observation—2^{nd} day (microscope, presence of cyanobacteria in the cytosol, presence of chlorophyll)
3^{rd} day—every 3 days, elimination from the culture medium of the carbon source, with constant lighting (12 h cycle) by 10% (100% to 90%, 90% to 81%, etc.): observation of photosynthesis, viability.

### C/ Donor 1—Host c

Standardised culture of the viable host Saccharomyces cerevisiae; standardised viable anaerobic culture of Chlorobium tepidum;
Irradiation of the donor (see preliminary tests above);
After a latent period of 4 hours (time for the number of survivors to quadruple, 2 generations), polyethylene glycol (PEG) induced fusion with Saccharomyces cerevisiae cells (donor: host ratio=5-10:1);
After fusion, electrical stimulation of membrane fusion (1 kV/cm of the depth of the culture, 2 pulses).

Observations:
After fusion, all the cultures were placed under lighting (sunlight spectrum) with a circadian rhythm (12 h of light: 12 h of darkness)
1^{st} observation—1^{st} day (microscope, presence of cyanobacteria in the cytosol, presence of chlorophyll)
2^{nd} observation—2^{nd} day (microscope, presence of cyanobacteria in the cytosol, presence of chlorophyll)
3^{rd} day—every 3 days, elimination from the culture medium of the carbon source, with constant lighting (12 h cycle) by 10% (100% to 90%, 90% to 81%, etc.): observation of photosynthesis, viability.

### D/ Donor 1—Host d

Viable standardised culture of the host Saccharomyces uvarum which had undergone HU pre-treatment (see preliminary tests above); standardised viable anaerobic culture of Chlorobium tepidum;
Irradiation of the donor (see preliminary tests above);

### Experiment 1D/

After a latent period of 4 hours (time for the number of survivors to quadruple, 2 generations), polyethylene glycol (PEG) induced fusion with Saccharomyces uvarum cells (donor: host ratio=5-10:1);
After fusion, electrical stimulation of membrane fusion (1 kV/cm of the depth of the culture, 2 pulses).

### Experiment 2D/

After a latent period of 4 hours (time for the number of survivors to quadruple, 2 generations), microinjection into the largest Saccharomyces uvarum cells;
2 doses: a) 10-15 donor cells; b) 5-10% of the volume of the host cells: (culture of surviving donors after a latent period).

Observations:
After fusion, all the cultures were placed under lighting (sunlight spectrum) with a circadian rhythm (12 h of light: 12 h of darkness)
1^{st} observation—1^{st} day (microscope, presence of cyanobacteria in the cytosol, presence of chlorophyll)
2^{nd} observation—2^{nd} day (microscope, presence of cyanobacteria in the cytosol, presence of chlorophyll)
3^{rd} day—every 3 days, elimination from the culture medium of the carbon source, with constant lighting (12 h cycle) by 10% (100% to 90%, 90% to 81%, etc.): observation of photosynthesis, viability.

### E/ Donor 2—Host a

Standardised culture of Cyanophora paradoxa;
Irradiation of the donor (see preliminary tests above);
After a latent period and verification of viability: isolation of the plast and nucleus fraction+verification of the intact membranes under the microscope.

### Experiment 1E/

Microinjection of a cyanelle fraction (membranes preserved, 5-10% of the volume of the host cells)

### Experiment 2E/

Microinjection of a cyanelle and nucleus fraction (5-10% of the volume of the host cells)

Observations:
1^{st} observation—32 cell stage
2^{nd} observation—tadpole (gills)
3^{rd} observation—tadpole (lungs)
4^{th} observation—adult frog (in vivo, histological analysis).

### F/ Donor 2—Host b

Standardised culture of Cyanophora paradoxa;
Irradiation of the donor (see preliminary tests above);
After a latent period (time for the number of survivors to quadruple, 2 generations, verification of viability under the microscope): fragilisation of the donor with lysozyme (see preliminary tests above);
Polyethylene glycol (PEG) induced fusion with mouse embryonic stem cells (donor: host ratio=5-10:1);
After fusion, electrical stimulation of the membrane fusion (1 kV/cm of the depth of the culture, 2 pulses).

Observations:
After fusion, all the cultures were placed under lighting (sunlight spectrum) with a circadian rhythm (12 h of light: 12 h of darkness)
1^{st} observation—1^{st} day (microscope, presence of cyanobacteria in the cytosol, presence of chlorophyll)
2^{nd} observation—2^{nd} day (microscope, presence of cyanobacteria in the cytosol, presence of chlorophyll)
3^{rd} day—every 3 days, elimination from the culture medium of the carbon source, with constant lighting (12 h cycle) by 10% (100% to 90%, 90% to 81%, etc.): observation of photosynthesis, viability.

### G/ Donor 2—Host c

Standardised culture of Cyanophora paradoxa;
Irradiation of the donor;
After a latent period (time for the number of survivors to quadruple, 2 generations, verification of viability under the microscope): fragilisation of the donor with lysozyme (see preliminary tests above);
Polyethylene glycol (PEG) induced fusion with Saccharomyces cerevisiae cells (donor: host ratio=5-10:1);
After fusion, electrical stimulation of the membrane fusion (1 kV/cm of the depth of the culture, 2 pulses).

Observations:
After fusion, all the cultures were placed under lighting (sunlight spectrum) with a circadian rhythm (12 h of light: 12 h of darkness)
1^{st} observation—1^{st} day (microscope, presence of cyanobacteria in the cytosol, presence of chlorophyll)
2^{nd} observation—2^{nd} day (microscope, presence of cyanobacteria in the cytosol, presence of chlorophyll)
3^{rd} day—every 3 days, elimination from the culture medium of the carbon source, with constant lighting (12 h cycle) by 10% (100% to 90%, 90% to 81%, etc.): observation of photosynthesis, viability.

### H/ Donor 2—Host d

Viable standardised culture of the host Saccharomyces uvarum which had undergone HU pre-treatment (see preliminary tests above);
Irradiation of the donor (see preliminary tests above);
After a latent period (time for the number of survivors to quadruple, 2 generations, verification of viability under the microscope): fragilisation of the donor with lysozyme (see preliminary tests above)

### Experiment 1H/

Polyethylene glycol (PEG) induced fusion with Saccharomyces uvarum cells (donor: host ratio=5-10:1);
After fusion, electrical stimulation of the membrane fusion (1 kV/cm of the depth of the culture, 2 pulses).

### Experiment 2Ha/

Irradiation of the donor (see preliminary tests above);
After a latent period (time for the number of survivors to quadruple, 2 generations), microinjection of the standardised suspension of a cyanelle fraction into the largest Saccharomyces uvarum cells;
5-10% of the volume of the host cells (culture of surviving donors after a latent period).

### Experiment 2Hb/

Irradiation of the donor (see preliminary tests above);
After a latent period (time for the number of survivors to quadruple, 2 generations), microinjection of the standardised suspension of the cyanelle+nucleus fraction of the donor (50:50) into the largest Saccharomyces uvarum cells;
5-10% of the volume of the host cells (culture of surviving donors after a latent period).

Observations:
After fusion, all the cultures were placed under lighting (sunlight spectrum) with a circadian rhythm (12 h of light: 12 h of darkness)
1^{st} observation—1^{st} day (microscope, presence of cyanobacteria in the cytosol, presence of chlorophyll)
2^{nd} observation—2^{nd} day (microscope, presence of cyanobacteria in the cytosol, presence of chlorophyll)
3^{rd} day—every 3 days, elimination from the culture medium of the carbon source, with constant lighting (12 h cycle) by 10% (100% to 90%, 90% to 81%, etc.): observation of photosynthesis, viability.

### I/ Donor 3—Host a

Standardised viable culture of Ostreococcus tauri;
Irradiation of the donor (see preliminary tests above);
After a latent period (time for the number of survivors to quadruple, 2 generations), fragilisation of donor cells by sonication;
Microinjection into a frog 1 cell zygote;
2 doses: a) 10-15 donor cells; b) 5-10% of the volume of the host cells: (culture of surviving donors after a latent period).

Observations:
1^{st} observation—32 cell stage
2^{nd} observation—tadpole (gills)
3^{rd} observation—tadpole (lungs)
4^{th} observation—adult frog (in vivo, histological analysis)

### J/ Donor 3—Host b

Standardised viable culture of Ostreococcus tauri;
Irradiation of the donor (see preliminary tests above);
After a latent period (time for the number of survivors to quadruple, 2 generations), fragilisation of donor cells by sonication;

### Experiment 1J/

Polyethylene glycol (PEG) induced fusion with mouse embryonic stem cells (donor: host ratio=5-10:1);
After fusion, electrical stimulation of membrane fusion (1 kV/cm of the depth of the culture, 2 pulses).

### Experiment 2J/

Microinjection into mouse embryonic stem cells;
2 doses: a) 10-15 donor cells; b) 5-10% of the volume of the host cells: (culture of surviving donors after a latent period).

Observations:
After fusion, all the cultures were placed under lighting (sunlight spectrum) with a circadian rhythm (12 h of light: 12 h of darkness)
1^{st} observation—1^{st} day (microscope, presence of cyanobacteria in the cytosol, presence of chlorophyll)
2^{nd} observation—2^{nd} day (microscope, presence of cyanobacteria in the cytosol, presence of chlorophyll)
3^{rd} day—every 3 days, elimination from the culture medium of the carbon source, with constant lighting (12 h cycle) by 10% (100% to 90%, 90% to 81%, etc.): observation of photosynthesis, viability.

### K/ Donor 3—Host c

Standardised viable culture of Ostreococcus tauri;
Irradiation of the donor (see preliminary tests above);
After a latent period (time for the number of survivors to quadruple, 2 generations), fragilisation of donor cells by sonication;
Polyethylene glycol (PEG) induced fusion with Saccharomyces cerevisiae cells (donor: host ratio=5-10:1);
After fusion, electrical stimulation of membrane fusion (1 kV/cm of the depth of the culture, 2 pulses).

Observations:
After fusion, all the cultures were placed under lighting (sunlight spectrum) with a circadian rhythm (12 h light: 12 h of darkness)
1^{st} observation—1^{st} day (microscope, presence of cyanobacteria in the cytosol, presence of chlorophyll)
2^{nd} observation—2^{nd} day (microscope, presence of cyanobacteria in the cytosol, presence of chlorophyll)
3^{rd} day—every 3 days, elimination from the culture medium of the carbon source, with constant lighting (12 h cycle) by 10% (100% to 90%, 90% to 81%, etc.): observation of photosynthesis, viability.

### L/ Donor 3—Host d

Viable standardised culture of the host Saccharomyces uvarum which had undergone HU pre-treatment (see preliminary tests above);
Standardised viable culture of Ostreococcus tauri;
Irradiation of the donor (see preliminary tests above);
After a latent period (time for the number of survivors to quadruple, 2 generations): fragilisation of donor cells by sonication

### Experiment 1L/

Polyethylene glycol (PEG) induced fusion with Saccharomyces uvarum cells (donor: host ratio=5-10:1);
After fusion, electrical stimulation of membrane fusion (1 kV/cm of the depth of the culture, 2 pulses).

### Experiment 2L/

Irradiation of the donor (see preliminary tests above);
After a latent period (time for the number of survivors to quadruple, 2 generations), microinjection of the standardised suspension of Ostreococcus tauri into the largest Saccharomyces uvarum cells;
If not toxic, 5-10% of the volume of the host cells (culture of surviving donors after a latent period).

It should be noted that similar experiments can be performed, by applying the procedures described above, in the presence of the various hosts mentioned, with the symbiont Rhodobacter sphaeroides, an autotrophic bacterium acting as donor.

### II/ Illustration for the Production of Ascorbic Acid

The present invention will be further illustrated by the production of ascorbic acid in an exosymbiosis ‘artificially’ established according to the method of this invention.

As a reminder, ascorbic acid or vitamin C is synthesised by many prokaryotes and a large majority of plants and animals Nevertheless, certain birds, guinea-pigs, the great apes and in particular humans are exceptions. Their inability to perform this synthesis is due to an inactive ψ GULO pseudogene, responsible for the production of the last enzyme in the biosynthetic chain, L-gulonolactone oxidase.

The objective is thus to correct the consequences of genetic deficiency in the host by ‘building’ a genetically modified symbiont, and thus, change the phenotype of the host in a controlled manner.

For this example, this was achieved using a method with various steps:
A/ Identifying and selecting the genes involved in ascorbic acid (vitamin C) biosynthesis

B/ Cloning the selected genes in the chromosome of the probiotic bacterium Escherichia coli Nissle 1917 strain, or possibly in any other gastrointestinal prokaryote symbiont of the guinea-pig, which here is the host with deficient ascorbic acid (vitamin C) biosynthesis

C/ Establishing the symbiosis of the genetically modified symbiont inside the guinea-pig host. Thus, the biosynthesis of vitamin C by the symbiont compensates for the natural incapacity of the host to perform this synthesis and this symbiosis prevents scurvy if the diet is deficient in ascorbic acid.

### A/ Identifying and Selecting the Genes Involved in Ascorbic Acid (Vitamin C) Biosynthesis)

The genes for the biosynthesis of L-ascorbate can be obtained in different ways, e.g.
Isolation of the metabolic pathway in Mycobacterium tuberculosis
Induction of the cluster of genes responsible for vitamin C biosynthesis in Xanthomonas campestris by oxidative stress, then identifying and cloning them
Activation and modification of metabolic pathways already existing in Escherichia coli by genetic engineering.

### B/ Cloning the Selected Genes in the Chromosome of the Probiotic Bacterium Escherichia coli Nissle 1917 Strain, or Possibly in any other Gastrointestinal Prokaryote Symbiont of the Guinea-Pig

The clusters of foreign genes were then integrated into the attachment site of the λ phage of the E. coli chromosome by prior association, by molecular cloning, of the biosynthesis genes in a vector with an λ-att site and an INT gene. In this way, the genes were integrated without damaging the host and it is stable because of the absence of the λ-xis gene (needed for excision).

In addition, the cells can be identified from the GFP (Green Fluorescence Protein) coded by the chromosome.

The colonies producing ascorbic acid were selected first by detecting a halo around the individual colonies on McConkey agar plates, using a technique well known to those working in the field.

The success of the genetic modification was checked by testing those positive for ascorbate in the culture medium (CosmoBioCo Ltd. Vitamin C Assay).

If vitamin C production is too low, the key gene or genes responsible for ascorbate breakdown can also be inactivated, e.g. by targeted deletion.

In addition if necessary, the most appropriate colonies producing ascorbate are selected, i.e. those which have a profile with a balance between ascorbate production and compatibility with the host's gastrointestinal pH environment.

### C/ Establishing the Symbiosis of the Genetically Modified Symbiont inside the Guinea-pig Host and Preventing Scurvy if the Diet is Deficient in Ascorbic Acid

Next, the modified symbiont (E. coli Nissle 1917 strain or another intestinal symbiont of the guinea-pig) was introduced into the host (guinea-pig) using a per-rectal enema and/or a gastric tube.

In vivo maintenance of the symbiont culture was tested and monitored by examining the excrement by fluorescence microscopy.

The anti-scurvy activity of the symbiont was tested using the following experiment:

Groups of animals were created and subjected to a vitamin C deficient diet:
1/ Control group 1 (with a normal intestinal flora)
2/ Control group 2 (with unmodified E. coli Nissle 1917 strain)
3/ Test group 1 (with E. coli Nissle 1917 strain producing ascorbate)
4/ Test group 2 (new-born guinea-pigs inoculated with E. coli Nissle 1917 strain)

They were evaluated by observing the symptoms of scurvy. As expected, from 4 weeks, the control groups developed scurvy, while the test groups remained asymptomatic.

In conclusion, there are many effects of new symbiotic relationships established using the method according to the invention which can be exploited, including:
Increasing and/or facilitating the host
Manipulation and/or control of the host
Curing and/or helping recovery of the host
Immunomodulation of the host
Improvement in the host's health
Decline in the host's health
Encouraging parthenogenesis in a host and/or production of a host organism and/or of host tissue clones, including the proliferation of tissues (adult animals producing their own clones, regeneration of tissues, etc.);
Secretion of hormones and/or of biocatalysts and/or neurotransmitters and/or any other biologically active compound of interest in a host
Changing environmental parameters because of the symbiotic relationship (e.g. as in environmental detoxication, biological recycling, terraformation, etc.)
Production of industrial compounds and/or oil and/or food (including dietary additives and constituents) derived from established symbiotic relationships

1-7. (canceled)

8. A method for establishing a symbiosis comprising the steps of:
selecting an organism or organelle to act as a symbiont and an organism to form a host, wherein the organism or organelle to act as the symbiont and the organism to form the host do not naturally exist in a symbiotic relationship;
bringing the symbiont and the host together into contact; and
maintaining an association between the symbiont and the host,
wherein the symbiont and/or the host have been subjected to genetic modification wherein the genetic modification contributes to the establishment and/or maintenance of the symbiosis before the symbiont and the host are brought into contact.

9. The method of claim 8 wherein the symbiont is not selected from the group consisting of: nitrogen fixing bacteria, endophytic fungi, and mitochondria with more than 90% sequence identity with the host's mitochondria.

10. The method of claim 8 wherein the genetic modification results from mutagenesis or transfer of genetic material.

11. The method of claim 8 wherein the association of the symbiont and the host is subjected to selection and/or maintenance pressure.

12. The method of claim 8 wherein the symbiont is selected from the group consisting of: Chlorobium tepidum, cyanelles from Cyanophora paradoxa, Ostreococcus tauri, and Rhodobacter sphaeroides.

13. The method of claim 8 wherein the host is selected from the group consisting of: frog zygotes (Xenopus tropicalis albinos), albino nude mice embryonic stem cells, Saccharomyces cerevisiae, and Saccharomyces uvarum.

14. The method of claim 8 wherein an endosymbiosis or an exosymbiosis is established.
